(19) United States
(12) Patent Application Publication
(10) Pub. No.: US 2025/0134619 A1
(43) Pub. Date: May 1, 2025

(54) RADIOPAQUE INK FORMULATION

(71) Applicant: Project Glow Holdings, LLC, Clawson, MI (US)

(72) Inventors: Matthew Osher, Novi, MI (US); Drew Steeh, Bloomfield Hills, MI (US)

(73) Assignee: Project Glow Holdings, LLC, Clawson, MI (US)

(21) Appl. No.: 18/926,810

(22) Filed: Oct. 25, 2024

Related U.S. Application Data
(60) Provisional application No. 63/593,183, filed on Oct. 25, 2023.

(51) Int. Cl. A61B 90/00; A61K 31/202; A61K 33/44; A61M 37/00

(52) U.S. Cl. CPC A61B 90/39; A61K 31/202; A61K 33/44; A61M 37/0084; A61B 2090/3912; A61B 2090/392; A61B 2090/3937; A61B 2090/3966; A61M 2205/32; A61M 2210/1021

(57) ABSTRACT

A tattoo ink formulation for endoscopic marking includes an ink solution having carbon suspended in water and a radiopaque solution having an ethiodized oil. The ink formation is configured to be viewable in both visible light and with non-invasive medical imaging, such as x-ray imaging and computed tomography (CT) scans.

### CROSS-REFERENCE TO RELATED APPLICATION

This application claims benefit and priority under 35 U.S.C. § 119(e) to U.S. Provisional Patent Application No. 63/593,183, filed Oct. 25, 2023, the disclosure of which is considered part of this application and is hereby incorporated by reference in its entirety.

### TECHNICAL FIELD

The present disclosure relates generally to radiopaque ink formulations and methods of marking a site, and in particular a gastrointestinal site, with the radiopaque ink formulation.

### BACKGROUND

Endoscopic tattooing typically involves injecting carbon ink into tissue to visually mark areas of the tissue, such as lesions or other irregularities. The markings may be used to provide later endoscopic examination of suspicious lesions or for marking for later surgical localization. It is also well known to use non-invasive medical imaging for surgical planning, patient consultation, and follow-up checks. However, gastrointestinal markings provided by traditional endoscopic tattooing are not visible with non-invasive medical imaging, making further endoscopic procedures necessary for viewing and monitoring previously marked internal sites.

### SUMMARY

This disclosure provides a tattoo ink formulation for endoscopic marking and associated devices and methods of marking a site such as a gastrointestinal site, with a radiopaque tattoo. The tattoo ink may include an ink solution having carbon suspended in water. The tattoo ink may also include a radiopaque solution having an ethiodized oil that is dispersed throughout the ink solution. The ink formulation is configured to be viewable in both visible light and with non-invasive medical imaging due to the combination of the ink solution and the radiopaque solution. The method of marking a gastrointestinal site involves providing a first volume of a carbon ink solution and a second volume of a radiopaque solution in a syringe. The carbon ink solution is mixed with the radiopaque solution to provide the radiopaque ink formulation in the syringe. An injection device is inserted into a target area in a gastrointestinal track and a desired portion of the radiopaque ink formulation is injected into the target area.

One aspect of the disclosure provides a tattoo ink formulation for gastrointestinal marking. The tattoo ink formulation includes an ink solution having carbon suspended in water and a radiopaque solution having an ethiodized oil. The ink formulation is configured to be viewable in both visible light and with non-invasive medical imaging, such as x-ray imaging and computed tomography (CT) scans.

In some examples, the ethiodized oil includes iodine and ethylated ester. The ethiodized oil may include a concentration of iodine in a range of approximately between 450 mg and 550 mg of iodine per milliliter of the radiopaque solution. The ethiodized oil may include a concentration of iodine of approximately 480 mg of iodine per milliliter of radiopaque solution.

In some implementations, the ethiodized oil comprises ethylated ester deprived from poppy seed oil. The ethylated ester may include a linoleic acid, an olein acid, a palmitic acid, and a stearic acid. The linoleic acid may comprise approximately 70% or more of the concentration of the ethylated ester. The linoleic acid may comprise approximately 73% of the concentration of the ethylated ester. The olein acid may comprise approximately 14% of the ethylated ester. The palmitic acid may comprise approximately 9% of the ethylated ester. The stearic acid may comprise approximately 3% of the ethylated ester.

The radiopaque solution, in some examples, has approximately 5% or less of the concentration of the tattoo ink formulation by mass. The radiopaque solution may also comprise approximately 4% of the concentration of the tattoo ink formulation by mass.

Another aspect of the disclosure provides a device for gastrointestinal tattooing that includes a syringe containing a premixed solution. The device may include an injection device configured to deliver the premixed solution to a target area in a gastrointestinal track for tattooing the target location. The premixed solution may include a first composition and a second composition. The first composition may be an ink solution. The second composition may be a radiopaque solution. The premixed solution includes a concentration of approximately 4% of the second composition by mass.

The first composition may include a carbon-based ink. The carbon-based ink may have a concentration of at least 90% of the premixed solution by mass. The second composition may include iodinated and ethylated ester derived from poppy seed oil. The second composition includes a concentration in a range approximately between 450 mg and 550 mg of iodine per milliliter of the second composition. The second composition includes a concentration of approximately 480 mg of iodine per milliliter of the second composition. The ethylated ester includes a linoleic acid, an olein acid, a palmitic acid, and a stearic acid. The linoleic acid may comprise approximately 70% or more of the concentration of the ethylated ester. The linoleic acid may comprise approximately 73% of the concentration of the ethylated ester. The olein acid may comprise approximately 14% of the ethylated ester. The palmitic acid may comprise approximately 9% of the ethylated ester. The stearic acid may comprise approximately 3% of the ethylated ester.

Yet another aspect of the disclosure provides a method of marking a gastrointestinal site with a radiopaque tattoo that involves providing a first volume of a carbon ink solution in a syringe, providing a second volume of a radiopaque solution in the syringe, and mixing the carbon ink solution and the radiopaque solution to provide a radiopaque ink in the syringe. The method further includes coupling an injection device into a target area in a gastrointestinal track and directing a portion of the radiopaque ink into the target area.

In some examples, the radiopaque ink may include iodine and an ethylated ester. The iodine may include a concentration in a range approximately between 450 mg and 550 mg of iodine per milliliter of radiopaque solution. The ethylated ester may contain a composition having 70% or more of linoleic acid. The radiopaque solution may have a concentration of less than 5% by mass of the radiopaque ink.

Each of the above independent aspects of the present disclosure, and those aspects described in the detailed description below, may include any of the features, options, and possibilities set out in the present disclosure and figures, including those under the other independent aspects, and may also include any combination of any of the features, options, and possibilities set out in the present disclosure and figures.

The details of one or more implementations of the disclosure are set forth in the accompanying drawings and the description below. Other aspects, advantages, purposes, and features will be apparent upon review of the following specification in conjunction with the drawings.

### BRIEF DESCRIPTION OF THE DRAWINGS

FIG. 1A is a camera image of the ink formulation with 0.05 mL of the radiopaque solution injected in an implant.

FIG. 1B is a camera image of the ink formulation with 0.02 mL of the radiopaque solution injected in an implant.

FIG. 1C is a camera image of the ink formulation with 0.005 mL of the radiopaque solution injected in an implant.

FIG. 2A is an x-ray image of the ink formulation without the radiopaque solution injected in an implant.

FIG. 2B is an x-ray image of the ink formulation with 0.02 mL of the radiopaque solution injected in the implant, as shown in FIG. 1B.

FIG. 2C is an x-ray image of the ink formulation with 0.005 mL of the radiopaque solution injected in the implant, as shown in FIG. 1C.

FIG. 3A is a CT scan image of the ink formulation with 0.05 mL of the radiopaque solution injected in the implant, as shown in FIG. 1A.

FIG. 3B is a CT scan image of the ink formulation with 0.02 mL of the radiopaque solution injected in the implant, as shown in FIG. 1B.

FIG. 3C is a CT scan image of the ink formulation with 0.005 mL of the radiopaque solution injected in the implant, as shown in FIG. 1C.

FIG. 3D is a CT scan image of the ink formulation without the radiopaque solution injected in an implant.

FIG. 4 is an exemplary device for injecting a radiopaque tattoo.

FIG. 5 is a flowchart illustrating a method of marking a gastrointestinal site with a radiopaque tattoo.

Like reference numerals indicate like parts throughout the drawings.

### DETAILED DESCRIPTION

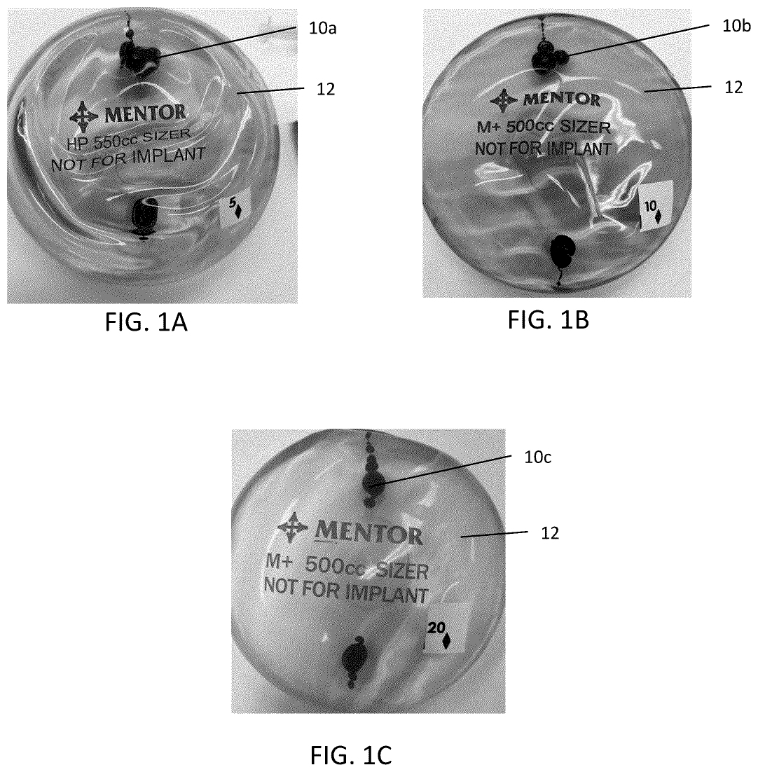

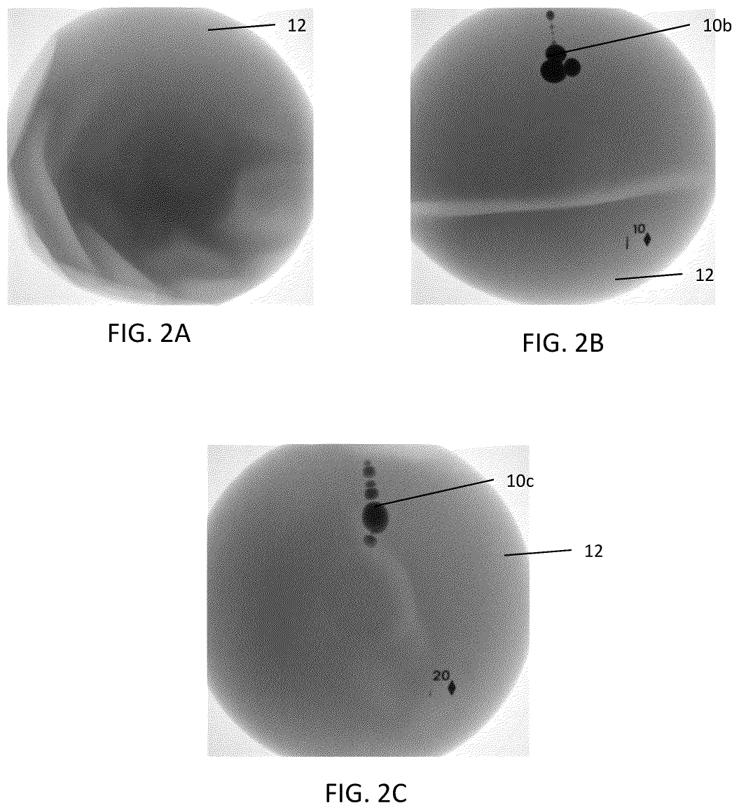

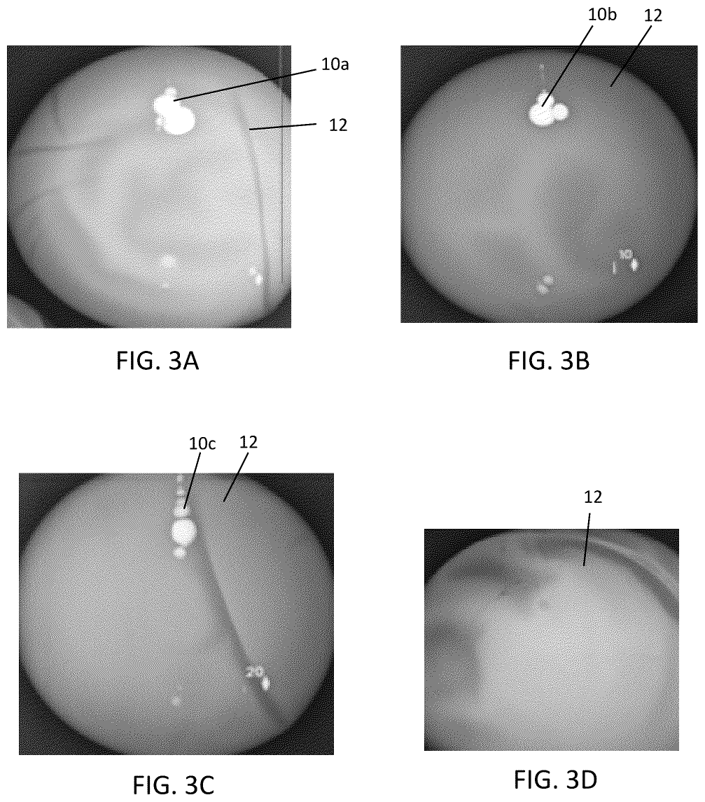

A radiopaque ink formulation and methods for marking a site with the radiopaque ink formulation are disclosed herein in various implementations. For example, the radiopaque ink formulation generally includes a first composition which is visible to the human eye, such as shown in FIGS. 1A-1C, and a second composition which is visible by various medical imaging techniques such as those using ionizing radiation, including x-ray imaging as shown in FIGS. 2A-2C and CT scans as shown in FIGS. 3A-3C. The ink formulation may be injected into a target area for endoscopic tattooing. In one example, the ink formulation may be injected into a target area of a gastrointestinal track for endoscopic tattooing.

The radiopaque tattoo ink formulation serves to localize and mark target areas, for example, suspicious lesions during endoscopy that require follow up or additional future management. The first composition may comprise an ink pigment visible to the human eye for anatomic localization during endoscopy procedures. The second composition may comprise a radiopaque composition visible via medical imaging for improved preoperative planning, patient consultation, and radiographic follow up to improve patient experiences and outcomes.

The tattoo ink formulation may include an ink solution and a radiopaque solution mixed together. The ink solution may include, for example, a pigment suspended in a liquid solution. The pigment is configured to act as a visible marking agent which is visible to the human eye or via cameras. The pigment may be suspended in at least water. The water may be mixed with additional liquid additives such as glycerol, polysorbate 80, benzyl alcohol, simethicone hyaluronic acid, or isopropyl alcohol. For example, hyaluronic acid may be added to the liquid solution to help reduce migration of the ink solution in tissue over time. Accordingly, the ink solution may include a mixture of the pigment, water, glycerol, polysorbate 80, benzyl alcohol, simethicone hyaluronic acid, and isopropyl alcohol. The pigment may comprise of at least carbon black, such as to refer to the ink solution as a carbon-based ink. Alternatively, the ink solution may consist of or include pigments other than carbon black, such as India ink or the like.

The radiopaque solution may include an ethiodized oil. The ethiodized oil is configured to act as a radiopaque contrast agent which is visible under medical imaging. The ethiodized oil may be comprised of iodine and ethylated ester. The radiopaque solution may include a concentration of iodine in ranges of approximately between 400 mg to 600 mg (e.g., 450 mg to 550 mg, or 450 mg to 500 mg) of iodine per milliliter of radiopaque solution. These ranges are inclusive of their endpoints. In one example, the radiopaque solution may include a concentration of iodine of approximately 480 mg of iodine per milliliter of radiopaque solution.

The ethylated ester may be derived from the fatty acids of poppyseed oil. The ethylated ester may be comprised of linoleic acid, olein acid, palmitic acid, and stearic acid. The formulation of the ethylated ester may include by mass at least 70% or more of linoleic acid. The formulation of the ethylated ester may include by mass at least 70% linoleic acid, a range of approximately between 10% to 15% of olein acid, 5% to 10% of palmitic acid, and 1% to 5% stearic acid. These ranges are inclusive of their endpoints. In an example, the ethylated ester may include by mass 73% linoleic acid, 14% olein acid, 9% palmitic acid, and 3% stearic acid.

The ink formulation may include the radiopaque solution in a concentration of less than 10% by mass, such as less than 5% by mass. In one example, the ink formulation may include the radiopaque solution in a concentration of 4% by mass. For example, a 10 cubic centimeter (cc) syringe or other container of the ink formulation may include less than 1 mL of radiopaque solution (e.g., less than 0.5 grams of radiopaque solution, for example, 0.4 mL of radiopaque solution). A 5 cc syringe or other container of the ink formulation may include 0.2 mL of the radiopaque solution.

Referring now to FIGS. 1A-1C, exemplary images of the ink formulation injected in a 500 cc breast implant 12 are shown for illustrating the visibility of the ink formulation in visible light and with medical non-invasive imaging. As illustrated in FIG. 1A, a radiopaque solution 10A in a 0.05 mL concentration is injected in the breast implant 12. As illustrated in FIG. 1B, a radiopaque solution 10B in a 0.02 mL concentration is injected in the breast implant 12. As illustrated in FIG. 1C, a radiopaque solution 10C in a 0.005 mL concentration is injected in the breast implant 12. As can be seen in FIGS. 1A-1C, the ink formulation is visible to the human eye or via cameras regardless of the provided ink concentrations to the visible ink solution. The contrast of the carbon black ink solution marks the injection site such that the marking is visible at the target site to the human eye or under a camera.

Referring to FIGS. 2A-2C, exemplary images of the breast implants 12 are illustrated under x-ray imaging for illustrating the visibility of the ink formulation under non-invasive medical imaging. As illustrated in FIG. 2A, an ink solution without a radiopaque solution is injected into the breast implant 12 and is not visible in the implant 12 under x-ray imaging. Thus, the carbon black ink solution alone is not visible under x-ray. Referring to FIG. 2B, the carbon black ink solution is provided with the radiopaque solution 10B in a 0.02 mL concentration injected in the breast implant 12. The radiopaque solution 10B is visible under the human eye due to the carbon black ink as illustrated in FIG. 1B. However it is also visible as illustrated in FIG. 2B under x-ray imaging due to the added radiopaque solution. The contrast of the ethiodized oil marks the injection site such that the marking is visible at the target site. Similarly, referring to FIG. 2C, the radiopaque solution 10C in a 0.005 ML concentration is shown as being visible under x-ray imaging due to the added radiopaque solution. Therefore, the radiopaque solution provides a solution for viewing the ink under visible light as illustrated in FIGS. 1A-1C and a solution for view the ink under non-invasive medical imaging such as x-ray imaging as illustrated in FIGS. 2B-2C. The ink itself without the added radiopaque solution is not visible under non-invasive medical imaging as shown in FIG. 2A.

Referring to FIGS. 3A-3C, exemplary images of the breast implants 12 are illustrated under CT scans for illustrating the visibility of the ink formulation under non-invasive medical imaging. As illustrated in FIG. 3D, an ink solution without a radiopaque solution is injected into the breast implant 12 and is not visible in the implant 12 under CT scans. Thus, the carbon black ink solution alone is not visible under CT scans. Referring to FIG. 3A, the carbon black ink solution is provided with the radiopaque solution 10A in a 0.05 mL concentration injected in the breast implant 12. The radiopaque solution 10A is visible under the human eye due to the carbon black ink as illustrated in FIG. 1A. However, it is also visible as illustrated in FIG. 3A under CT imaging due to the added radiopaque solution. The contrast of the ethiodized oil marks the injection site such that the marking is visible at the target site. Similarly, referring to FIG. 3B, the radiopaque solution 10B in a 0.02 mL concentration is shown as being visible under CT imaging due to the added radiopaque solution, and referring to FIG. 3C, the radiopaque solution 10C in a 0.005 mL concentration is shown as being visible under CT imaging due to the added radiopaque solution. Therefore, the radiopaque solution provides a solution for viewing the ink under visible light as illustrated in FIGS. 1A-1C and a solution for viewing the ink under non-invasive medical imaging such as CT scans as illustrated in FIGS. 3A-3C. The ink itself without the added radiopaque solution is not visible under non-invasive medical imaging as shown in FIG. 3D.

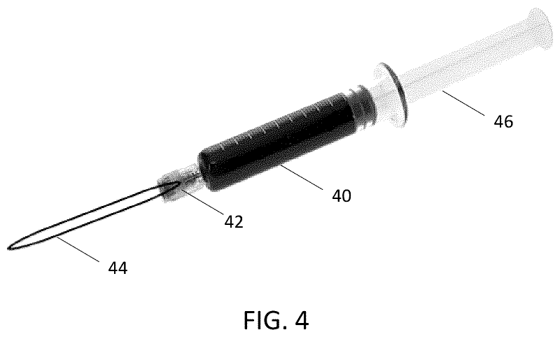

Referring to FIG. 4, an exemplary device for marking a site with a radiopaque tattoo ink is shown. A premixed solution comprising a first volume of a carbon ink solution and a first volume of a radiopaque solution may be provided in a syringe 40 or other container. The carbon ink solution and the radiopaque solution may be mixed in the syringe to create the radiopaque tattoo ink solution. The syringe 40 may be provided with an injection end 42 for coupling to an injection device 44 such as a needle for delivering the premixed solution to a target area. The syringe 40 may be provided with a plunger 46 opposite the injection end 42. Upon depression of the plunger 46, the premixed solution may be directed from the syringe 40 through the injection end 42 and the injection device 44 into a target area.

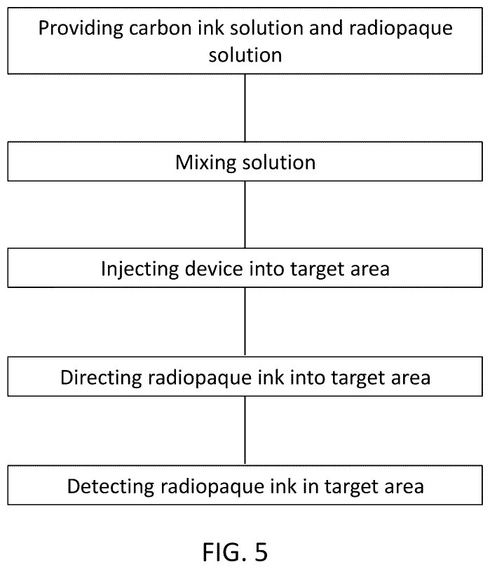

Referring to FIG. 5, a flow chart illustrating a method for marking a site with the radiopaque tattoo ink is shown. The radiopaque ink may be used for marking various locations which require endoscopic examination. In one example, the radiopaque ink may be used for marking gastrointestinal sites. In a first step, a first volume of a carbon ink solution is provided into a syringe or other container. For example, 5 mL of the carbon ink solution may be provided into a syringe. The carbon ink solution may comprise of a crushed carbon, such as carbon black, suspended in a liquid solution including at least water. The liquid solution may comprise of additional components such as glycerol, polysorbate 80, benzyl alcohol and simethicone. A second volume of a radiopaque solution is provided into the same syringe or container. For example, 0.2 mL of the radiopaque solution may be provided into the syringe. The radiopaque solution may comprise of an ethiodized oil containing iodine and ethylated ester. The radiopaque solution may include a concentration by mass of iodine in a range of approximately between 450 mg and 550 mg of iodine per milliliter of radiopaque solution. For example, the solution may include 480 mg of iodine per milliliter of radiopaque solution. The ethylated ester may include a concentration by mass of linoleic acid in a range of 70% to 80%. For example, the linoleic acid may comprise 73% of the ethylated ester. The first volume and the second volume are such that the radiopaque has a concentration of less than 5% by mass of the total of the first volume and the second volume.

In a second step, the first volume of the carbon ink solution and the second volume of the radiopaque solution are mixed to provide a radiopaque ink solution inside the syringe or container. The individual solutions may be first mixed during manufacture of the radiopaque ink solution as both individual solutions are provided in the syringe together. The solutions may undergo a second mixing during the procedure of endoscopic tattooing to ensure the individual solutions are properly mixed in the syringe prior to injection to counteract separation of the individual solutions that may have occurred post-production. The syringe having the carbon ink solution and the radiopaque solution may be premixed at production during the first mixing. For example, the carbon ink solution and the radiopaque solution may be mixed in the syringe during manufacture to produce the radiopaque ink solution. The second mixing may occur during the procedure of tattooing. For example, the syringe may be mixed via shaking prior to injection of the ink into tissue to ensure the radiopaque ink solution is reconstituted from any separation of the individual ink solution and the radiopaque solution prior to injection. In some examples, the carbon may be suspended in the ink solution and may require mixing prior to injection of the ink solution to disperse the carbon amongst the solution in a generally consistent dispersion to counteract any separation or sinking of the carbon that occurs after manufacture and prior to the procedure, for example during the shelf life of the solution. Therefore, the radiopaque ink solution may be prepared during manufacture via the initial mixing or combining of the carbon ink solution and the radiopaque solution in the syringe. Further mixing may occur at the time of the procedure and prior to injection to ensure the carbon ink solution and the radiopaque solution are thoroughly mixed in case the individual solutions have settled or separated post-production. Mixing of the solutions may occur via, for example, shaking of the syringe or using an agitator.

In a third step, the syringe or other container is coupled with an injection device such as an endoscopic needle. The injection device is configured to inject the radiopaque ink solution into a target area. The injection device may be injected into the target area. For example, the injection device may be injected into the submucosal layer of the bowel for endoscopic tattooing.

In a fourth step, the radiopaque ink solution may be directed into the target area. If provided in a syringe, a plunger may be depressed to inject the radiopaque ink solution into the submucosal layer via the injection device. Injections of the radiopaque ink solution may include several injections around the circumference of the bowel for endoscopic tattooing. For example, injections in a range approximately between 0.5 mL and 1.0 mL (e.g., 0.5 mL to 0.75 mL) may be injected into approximately four locations around the circumference of the bowl.

In a fifth step, once the ink has been injected, the radiopaque ink may be detected in the target areas. For example, the radiopaque solution may be visible to the human eye due to the carbon black ink solution or via non-invasive medical imaging techniques such as x-ray or CT scans due to the radiopaque solution. The carbon black ink solution may permanently remain in the submucosal layer as a permanent tattoo. The radiopaque solution, such as the ethiodized oil may remain in the submucosal layer for months prior to breaking down, thus allowing continued or detection of the solution for an extended period of time after initial injection. Injection of the radiopaque ink solution provides anatomic location and visualization on repeat endoscopy or surgical interventions as well as being identifiable on radiographic or computed tomography imaging to providing improved preoperative planning and consultation.

For purposes of this disclosure, the term “coupled” (in all of its forms, couple, coupling, coupled, etc.) generally means the joining of two components directly or indirectly to one another. Such joining may be stationary in nature or movable in nature; may be achieved with the two components and any additional intermediate members being integrally formed as a single unitary body with one another or with the two components; and may be permanent in nature or may be removable or releasable in nature, unless otherwise stated.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional implementations that also incorporate the recited features. Furthermore, the terms “first,” “second,” and the like, as used herein do not denote any order, quantity, or importance, but rather are used to denote element from another.

Numbers, percentages, ratios, or other values stated herein are intended to include that value, and also other values that are “about” or “approximately” the stated value, as would be appreciated by one of ordinary skill in the art encompassed by implementations of the present disclosure. A stated value should therefore be interpreted broadly enough to encompass values that are at least close enough to the stated value to perform a desired function or achieve a desired result. For example, the terms “approximately,” “about,” and “substantially” may refer to an amount that is within less than 5% of, within less than 1% of, within less than 0.1% of, and within less than 0.01% of a stated amount.

Further, it should be understood that any directions or reference frames in the preceding description are merely relative directions or movements. For example, the terms “upper,” “lower,” “right,” “left,” “rear,” “front,” “vertical,” “horizontal,” “inboard,” “outboard” and derivatives thereof shall relate to the orientation shown in FIG. 1. However, it is to be understood that various alternative orientations may be provided, except where expressly specified to the contrary. It is also to be understood that the specific devices and processes illustrated in the attached drawings, and described in this specification are simply exemplary embodiments of the inventive concepts defined in the appended claims. Hence, specific dimensions and other physical characteristics relating to the embodiments disclosed herein are not to be considered as limiting, unless the claims expressly state otherwise.

Changes and modifications in the specifically described embodiments may be carried out without departing from the principles of the present invention, which is intended to be limited only by the scope of the appended claims as interpreted according to the principles of patent law. The disclosure has been described in an illustrative manner, and it is to be understood that the terminology which has been used is intended to be in the nature of words of description rather than of limitation. Many modifications and variations of the present disclosure are possible in light of the above teachings, and the disclosure may be practiced otherwise than as specifically described.

1. A tattoo ink formulation for endoscopic marking, the tattoo ink formulation comprising:
an ink solution having carbon suspended in water; and
a radiopaque solution having an ethiodized oil, the ink formulation configured to be viewable in both visible light and with non-invasive medical imaging.

2. The tattoo ink formulation of claim 1, wherein the ethiodized oil comprises iodine and ethylated ester.

3. The tattoo ink formulation of claim 1, wherein the ethiodized oil includes a concentration of iodine between 450 mg and 550 mg of iodine per milliliter of the radiopaque solution.

4. The tattoo ink formulation of claim 1, wherein the ethiodized oil includes a concentration of iodine of 480 mg of iodine per milliliter of the radiopaque solution.

5. The tattoo ink formulation of claim 1, wherein the ethiodized oil comprises ethylated ester derived from poppy seed oil.

6. The tattoo ink formulation of claim 5, wherein the ethylated ester includes a linoleic acid, an olein acid, a palmitic acid, and a stearic acid.

7. The tattoo ink formulation of claim 6, comprising the linoleic acid of 70% or more of a concentration of the ethylated ester.

8. The tattoo ink formulation of claim 7, comprising the linoleic acid of 73% of the concentration of the ethylated ester, the olein acid of 14% of the ethylated ester, the palmitic acid of 9% of the ethylated ester, and the stearic acid of 3% of the ethylated ester.

9. The tattoo ink formulation of claim 1, wherein the radiopaque solution has a concentration of less than 5% by mass of the tattoo ink formulation.

10. The tattoo ink formulation of claim 1, wherein the radiopaque solution has a concentration of approximately 4% by mass of the tattoo ink formulation.

11. A device for endoscopic tattooing, comprising:
a syringe containing a premixed solution;
the premixed solution containing a first composition and a second composition; and
an injection device configured to deliver the premixed solution to a target area for tattooing the target area,
wherein the first composition is an ink solution and the second composition is a radiopaque solution.

12. The device of claim 11, wherein the first composition includes a carbon-based ink.

13. The device of claim 12, wherein the carbon-based ink has a concentration of at least 90% of the premixed solution by mass.

14. The device of claim 11, wherein the second composition includes iodinated and ethylated ester derived from poppy seed oil.

15. The device of claim 14, wherein the second composition includes a concentration of iodine between 450 mg and 550 mg of iodine per milliliter of the radiopaque solution.

16. The device of claim 14, wherein the second composition includes a concentration of iodine of approximately 480 mg of iodine per milliliter of the radiopaque solution.

17. The device of claim 14, wherein the ethylated ester includes a linoleic acid, an olein acid, a palmitic acid, and a stearic acid.

18. The device of claim 17, comprising the linoleic acid of 70% or more of a concentration of the ethylated ester.

19. The device of claim 18, comprising the linoleic acid of 73% of the concentration of the ethylated ester, the olein acid of 14% of the ethylated ester, the palmitic acid of 9% of the ethylated ester, and the stearic acid of 3% of the ethylated ester.

20. A method of marking a target site with a radiopaque tattoo, comprising:
providing a first volume of a carbon ink solution in a syringe;
providing a second volume of a radiopaque solution in the syringe;
mixing the carbon ink solution and the radiopaque solution to provide a radiopaque ink formulation in the syringe;
coupling an injection device with the syringe;
injecting the injection device into a target area; and
directing a portion of the radiopaque ink formulation into the target area,
wherein the radiopaque solution includes iodine and an ethylated ester,
wherein the radiopaque solution includes a concentration of between 450 mg and 550 mg of the iodine per milliliter of the radiopaque solution,
wherein 70% or more of a concentration of the ethylated ester is linoleic acid, and
wherein the radiopaque solution has a concentration of less than 5% by mass of the radiopaque ink formulation.
